(19) United States
(12) Patent Application Publication
(10) Pub. No.: US 2006/0030538 A1
(43) Pub. Date: Feb. 9, 2006

(54) Methods for reducing or preventing localized fibrosis using SiRNA

(75) Inventor: Marc Hendriks, Brussum (NL)

Correspondence Address: MUETING, RAASCH & GEBHARDT, P.A., P.O. BOX 581415, MINNEAPOLIS, MN, 55458, US

(73) Assignee: Medtronic, Inc., Minneapolis, MN (US)

(21) Appl. No.: 11/183,486

(22) Filed: Jul. 18, 2005

Related U.S. Application Data
(60) Provisional application No. 60/589,724, filed on Jul. 21, 2004.

(51) Int. Cl. A61K 48/00; C12N 15/87

(52) U.S. Cl. 514/44; 435/455

(57) ABSTRACT

Methods for reducing or preventing localized fibrosis in a localized tissue region using SiRNA technology.

### CROSS-REFERENCE TO RELATED APPLICATIONS

The present application claims the benefit of U.S. Provisional Application No. 60/589,724, filed on 21 Jul. 2004, which is incorporated herein by reference in its entirety.

### BACKGROUND

In collagen, crosslinking is initiated only after specific Lysine (Lys) or Hydroxylysine (Hyl) residues of the telopeptides are converted extracellularly by lysyl oxidase into the aldehydes allysine and hydroxyallysine, respectively. These aldehydes subsequently react with Lys, Hyl, or hystidyl residues of the triple helix.

There are two pathways of formation of crosslinks, depending on whether the residue in the telopeptide is a Lys (allysine route) or a Hyl (hydroxyallysine route). The aldehydes react with specific Lys or Hyl residues in the triple helical domain on juxtaposed neighboring molecules to form difunctional intermediates that mature into trifunctional crosslinks. The two pathways lead to different types of crosslinks. Only the difunctional crosslinks that result from the hydroxyallysine route are able to mature into the trifunctional crosslinks HP and LP.

It has recently been discovered that aberrant crosslinking of collagen, i.e., an increase of hydroxyallysine-derived crosslinks at the expense of allysine-derived crosslinks is a causative mechanism in the formation of fibrotic tissue, such as is seen in abnormal wound healing of the skin, such as in hypertrophic scarring, which contains large amounts of hydroxyallysine-derived crosslinks.

A predominance of these types of crosslinks is also found in collagen produced after wounding of the corneal stroma; the resulting scar shows markedly increased levels of hydroxyallysine derived crosslinks at the expense of allysine crosslinks. The studies on elevated hydroxyallysine-derived crosslinks in abnormal scarring have been confirmed, followed by reports on increased hydroxyallysine-derived crosslinks in other (mainly fibrotic) disorders, such as various lung diseases (respiratory distress syndrome, idiopathic pulmonary fibrosis, hypersensitivity pneumonitis, respiratory bronchiolitis, silicosis and bleomycin-induced lung fibrosis), chronic adriamycin nephropathy (an experimental model resulting in non-immunologic glomerulosclerosis and interstitial fibrosis), infarct scar of the myocardium, joint contractures, vessel luminal narrowing, lipodermatosclerosis, annulo-aortic ectasia, fibrotic lesions of Dupuytren's disease, skin of patients with lipoid proteinosis, diabetes, skin fibrosis due to chromoblastomycosis infection, skeletal muscle injury, tendon hypertrophy and various liver diseases such as in alveolar echinococcosis (a dense and irreversible fibrosis), hepatocellular carcinoma, alcoholic cirrhosis or cirrhotic livers induced by viral hepatitis or by Schistosoma mansoni. This aberrant crosslinking of collagen and the mechanism involving TLH enzyme and PLOD2 gene is described in U.S. Pat. Pub. 2003/0219852 (Bank et al.).

Collagen crosslinked through the hydroxyallysine route is more difficult to degrade than collagen crosslinked through the allysine route because the hydroxyallysine-based crosslinks are less susceptible to proteolytic degradation than collagen crosslinked by allysine-based residues. Bank et al. in U.S. Pat. Pub. 2003/0219852 concluded that one of the characteristics of fibrotic lesions is an upregulation of telopeptide lysyl hydroxylase (TLH). It has also been shown that PLOD2, the gene that encodes telopeptide lysyl hydroxylase, is highly expressed in cells associated with a variety of fibrotic disorders.

Thus, it remains desirable to find suitable methods that reduce, and even prevent, aberrant crosslinking of collagen and the consequent formation of fibrotic tissue.

### SUMMARY

The present invention is directed to methods that cause the production of collagen containing telopeptide lysine instead of telopeptide hydroxylysine, or alternatively the inhibition of telopeptide lysyl hydroxylase so as to enhance the formation of allysine crosslinks at the expense of hydroxyallysine crosslinks, in a subject to reduce the formation of fibrotic tissue. The methods of the present invention utilize siRNA technology. The methods of the present invention therefore involve reducing, and preferably eliminating, aberrant crosslinking of collagen (and the consequent formation of fibrotic tissue) using siRNA technology.

Thus, the present invention provides methods that suppress the production and/or activity of a TLH enzyme in collagen-producing cells. This can involve directly targeting a PLOD2 gene encoding a TLH enzyme, or a gene involved in the production or processing of a TLH enzyme.

In one embodiment, there is provided a method of reducing or preventing fibrotic tissue formation in a subject, the method includes delivering a polynucleotide that suppresses the production and/or activity of a TLH enzyme in collagen-producing cells, wherein the polynucleotide includes an siRNA molecule or DNA encoding an siRNA molecule, wherein the siRNA molecule interferes with a PLOD2 gene and inhibits the translation of a TLH enzyme, or interferes with a gene that encodes a protein involved in the production or processing of a TLH enzyme.

In one embodiment, the polynucleotide is embedded in a polynucleotide delivery matrix. The polynucleotide delivery matrix can include an siRNA molecule that interferes with a PLOD2 gene and inhibits the translation of a TLH enzyme or interferes with a gene encoding a protein involved in the production or processing of a TLH enzyme. Alterntatively, the polynucleotide delivery matrix can include DNA encoding an siRNA molecule that interferes with a PLOD2 gene and inhibits the translation of a TLH enzyme or interferes with a gene encoding a protein involved in the production or processing of a TLH enzyme.

In addition to using a polynucleotide delivery matrix, the siRNA molecules, or DNA encoding siRNA molecules, can be delivered to the desired site using a variety of other methods. Such methods include, for example, a subcutaneous, intradermal, intramuscular, intrathecal, intra-organ, intratumoral, intralesional, intravesicle, and intraperitoneal method of delivery.

Preferably, the polynucleotide is delivered to a localized tissue region. This can be accomplished through the use of a device selected from the group consisting of implantable pumps, delivery catheters, needles, microneedle arrays, devices for high-velocity particle implantation, or any other known method for introducing a composition into a localized tissue region (e.g., surgically implanted).

The phrase “suppresses the production and/or activity of TLH enzyme” refers to a DNA encoding siRNA molecules, or siRNA molecules per se that prevents or otherwise reduces the production of a TLH enzyme, or that prevents or otherwise reduces the activity of a TLH enzyme, or both affects the production and activity of a TLH enzyme.

Herein, when reference is made to a PLOD2 gene or a TLH enzyme, reference is typically being made to the sequences of the human gene and enzyme, but the sequences of PLOD2 genes and TLH enzymes of other species are included where appropriate. Such sequences are disclosed in the NCBI database on the World Wide Web at ncbi.nim.nig.gov and specifically at ncbi.nlm.nih.gov/entrez/query.fcgi?CMD=Display&DB=nucleotide.

By “subject” is meant an organism to which the active agent of the invention can be administered. Preferably, a subject is a mammal or mammalian cells, e.g., such as humans, cows, sheep, apes, monkeys, swine, dogs, cats, and the like. More preferably, a subject is a human.

As used herein “cell” is used in its usual biological sense, and does not refer to an entire multicellular organism. The cell may be present in an organism which may be a human but is preferably of mammalian origin, e.g., such as humans, cows, sheep, apes, monkeys, swine, dogs, cats, and the like. However, several steps of producing small interfering RNA may require use of prokaryotic cells (e.g., bacterial cell) or eukaryotic cell (e.g., mammalian cell) and thereby are also included within the term “cell.”

By “complementary” it is meant that a molecule including one or more polynucleotides (DNA or RNA) can form hydrogen bond(s) (i.e., hybridize and form a duplex) with another molecule including one or more polynucleotides by either traditional Watson-Crick pairing or other non-traditional types. It will be understood that a complementary nucleotide sequence includes, in addition to a fully complementary nucleotide sequence, a substantially complementary nucleotide sequence that contains deletions or additions of one or more bases relative to the reference sequence, provided the complementary nucleotide sequence still retains the ability to hybridize with the reference nucleotide sequence.

The term “expression” defines the process by which a gene is transcribed into RNA (transcription); the RNA may be further processed into the mature small interfering RNA.

The terminology “expression vector” defines a vector or vehicle designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

By the term “inhibit” or “inhibitory” it is meant that the activity of the target genes or level of mRNAs or equivalent RNAs encoding target genes is reduced below that observed in the absence of the provided small interfering RNA. Preferably the inhibition is at least 10% less, 25% less, 50% less, or 75% less, 85% less, or 95% less than in the absence of the small interfering RNA.

By “polynucleotide” as used herein is meant a molecule having nucleotides of any length, either ribonucleotides or deoxynucleotides. The term is often used interchangeably with nucleic acid or nucleic acid molecule. The polynucleotide can be single, double, or multiple stranded and may include modified or unmodified nucleotides or non-nucleotides or various mixtures and combinations thereof. It can include DNA or RNA. An example of a polynucleotide according to the invention is a gene that encodes for a small interfering RNA, even though it does not necessarily have its more common meaning for encoding for the production of protein.

By “RNA” is meant ribonucleic acid, a molecule consisting of ribonucleotides connected via a phosphate-ribose(sugar) backbone. By “ribonucleotide” is meant guanine, cytosine, uracil, or adenine or some a nucleotide with a hydroxyl group at the 2′ position of a beta-D-ribo-furanose moiety. As is well known in the art, the genetic code uses thymidine as a base in DNA sequences and uracil in RNA. One skilled in the art knows how to replace thymidine with uracil in a written polynucleotide to convert a written DNA sequence into a written RNA sequence, or vice versa.

By “small interfering RNA” is meant a polynucleotide which has complementarity in a substrate binding region to a specified gene target, and which acts to specifically guide enzymes in the host cell to cleave the target RNA. That is, the small interfering RNA by virtue of the specificity of its sequence and its homology to the RNA target, is able to cause cleavage of the RNA strand and thereby inactivate a target RNA molecule because it is no longer able to be transcribed. These complementary regions allow sufficient hybridization of the small interfering RNA to the target RNA and thus permit cleavage. One hundred percent complementarity often necessary for biological activity and therefore is preferred, but complementarity as low as 90% may also be useful in this invention. The specific small interfering RNA described in the present application are not meant to be limiting and those skilled in the art will recognize that all that is important in a small interfering RNA is that it have a specific substrate binding site which is complementary to one or more of the target nucleic acid regions.

Small interfering RNAs are double stranded RNA agents that have complementary to (i.e., able to base-pair with) a portion of the target RNA (generally messenger RNA). Generally, such complementarity is 100%, but can be less if desired, such as at least 91%, at least 92%, at least 93%, at least 94%, at least 95%, at least 96%, at least 97%, at least 98%, or at least 99%. For example, 19 bases out of 21 bases may be base-paired. In some instances, where selection between various allelic variants is desired, 100% complementary to the target gene is required in order to effectively discern the target sequence from the other allelic sequence. When selecting between allelic targets, choice of length is also an important factor because it is the other factor involved in the percent complementary and the ability to differentiate between allelic differences.

The small interfering RNA sequence needs to be of sufficient length to bring the small interfering RNA and target RNA together through complementary base-pairing interactions. The small interfering RNA of the invention may be of varying lengths. The length of the small interfering RNA is preferably greater than or equal to ten nucleotides and of sufficient length to stably interact with the target RNA; specifically 15-30 nucleotides; more specifically, any integer between 15 and 30 nucleotides, such as 15, 16, 17, 18, 19, 20, 21, 22, 23, 24, 25, 26, 27, 28, 29, and 30. By “sufficient length” is meant a polynucleotide of greater than or equal to 15 nucleotides that is of a length great enough to provide the intended function under the expected condition. By “stably interact” is meant interaction of the small interfering RNA with a target polynucleotide (e.g., by forming hydrogen bonds with complementary nucleotides in the target under physiological conditions). The terms “comprises” and variations thereof do not have a limiting meaning where these terms appear in the description and claims.

As used herein, “a,” “an,” “the,” “at least one,” and “one or more” are used interchangeably. Thus, for example, a composition that comprises “a” polynucleotide (e.g., DNA or siRNA) can be interpreted to mean that the composition includes “one or more” polynucleotides. Furthermore, a “composition” as used herein can consist of just one polynucleotide without any other components (e.g., pharmaceutically acceptable carrier).

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1. Allysine route (telopeptide Lys); characteristic of type I collagen in skin, cornea, and certain tendons.

FIG. 2. Hydroxyallysine route (telopeptide Hyl); characteristic of type I collagen in bone and type II collagen in cartilage.

FIG. 3. Graphical representation of the results of siRNA-based reduction of PLOD2 expression in human HeLa cells (Experiment 1). Results are being expressed as % relative PLOD2 expression. Compared to control, untransfected controls reduction of PLOD2 expression is 90%.

FIG. 4. Graphical representation of the results of siRNA-based reduction of PLOD2 expression in human HeLa cells (Experiment 2). Results are being expressed as % relative PLOD2 expression. Compared to control, untransfected controls reduction of PLOD2 expression is 90%.

FIG. 5. Relative PLOD2 expression in transfected fibroblasts.

FIG. 6. Graphical representation of the results of a study using scrambled siRNA on expression of the housekeeping gene GAPDH in human fibroblasts.

FIG. 7. Graphical representation of the results of a study using scrambled siRNA on PLOD2 expression in human fibroblasts.

FIG. 8. 8A) Graphical representation of siRNA-based PLOD2 expression in relation to the housekeeping gene B2M in primary human skin fibroblasts. 8B) Graphical representation of siRNA-based COL1A2 expression in relation to the housekeeping gene B2M in primary human skin fibroblasts.

FIG. 9. 9A) Graphical representation of siRNA-based PLOD2 expression in relation to COL1A2 expression in primary human skin fibroblasts. 9B) Graphical representation of siRNA-based PLOD1 expression in relation to COL1A2 expression in primary human skin fibroblasts. 9C) Graphical representation of siRNA-based PLOD3 expression in relation to COL1A2 expression in primary human skin fibroblasts.

FIG. 10. 10A) Graphical representation of siRNA-based COL3A1 expression in relation to COL1A2 expression in primary human skin fibroblasts. 10B) Graphical representation of siRNA-based LOX expression in relation to COL1A2 expression in primary human skin fibroblasts. 10C) Graphical representation of siRNA-based P4HA-1 expression in relation to COL1A2 expression in primary human skin fibroblasts.

FIG. 11. 11A) Graphical representation of siRNA-based PLOD2 expression in relation to the housekeeping gene B2M in rat skin fibroblasts. 11B) Graphical representation of siRNA-based PLOD1 expression in relation to the housekeeping gene B2M in rat skin fibroblasts. 11C) Graphical representation of siRNA-based PLOD3 expression in relation to the housekeeping gene B2M in rat skin fibroblasts. 11D) Graphical representation of siRNA-based COL1A2 expression in relation to the housekeeping gene B2M in rat skin fibroblasts. 11E) Graphical representation of siRNA-based COL3A1 expression in rat skin fibroblasts. 11F) Graphical representation of siRNA-based LOX expression in relation to the housekeeping gene B2M in rat skin fibroblasts. 11G) Graphical representation of siRNA-based P4HA-1 expression in relation to the housekeeping gene B2M in rat skin fibroblasts.

FIG. 12. 12A) Graphical representation of siRNA-based PLOD2 expression in relation to COL1A2 expression in rat skin fibroblasts. 12B) Graphical representation of siRNA-based PLOD1 expression in relation to COL1A2 expression in rat skin fibroblasts. 12C) Graphical representation of siRNA-based COL3A1 expression in relation to COL1A2 expression in rat skin fibroblasts. 12D) Graphical representation of siRNA-based LOX expression in relation to COL1A2 expression in rat skin fibroblasts. 12E) Graphical representation of siRNA-based P4HA-1 expression in relation to COL1A2 expression in rat skin fibroblasts.

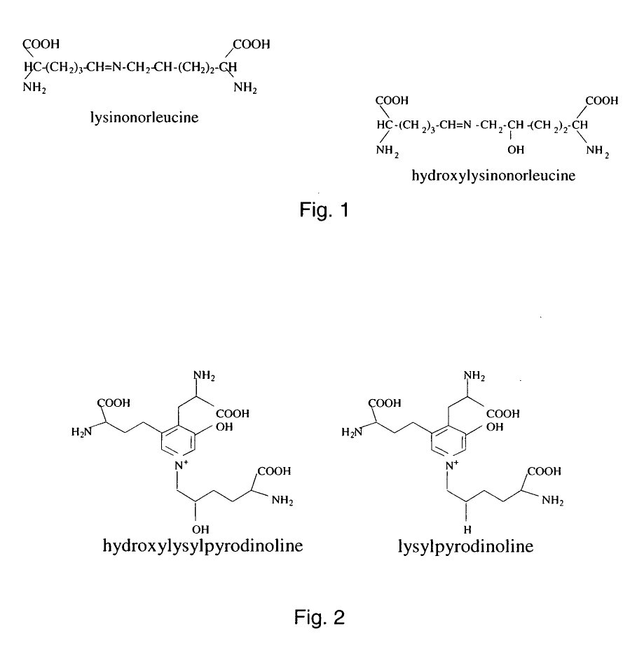

### DETAILED DESCRIPTION OF ILLUSTRATIVE EMBODIMENTS

The present invention is directed to methods that reduce or prevent the formation of fibrotic tissue in a subject. Such methods involve the use of siRNA technology to cause the production of collagen containing telopeptide lysine instead of telopeptide hydroxylysine, or alternatively the inhibition of telopeptide lysyl hydroxylase as to enhance the formation of allysine cross-links at the expense of hydroxyallysine crosslinks. The methods of the present invention therefore involve reducing, and preferably eliminating, aberrant crosslinking of collagen (and the consequent formation of fibrotic tissue) using siRNA technology.

Fibrotic tissue formation occurs in myocardial infarct related fibrosis, cardiac fibrosis, valvular stenosis, intimal hyperplasia, diabetic ulcers, peridural fibrosis, perineural fibrosis, radiation induced fibrosis, macular degeneration, or rhino-sinusitis related fibrosis. Accordingly, fibrotic tissue formation is associated with a wide variety of diseases and disorders, including, for example, heart rhythm disorder, heart failure, valve disease, vascular disease, diabetes, neurological diseases and disorders, or surgery. The SiRNA-based method of the present invention can be used to reduce or prevent fibrosis associated with such diseases and disorders.

SiRNA methodology involves the use of a polynucleotide such as DNA that encodes siRNA molecules or siRNA molecules per se to suppress the production and/or activity of a TLH enzyme in collagen-producing cells of a subject. The means by which a polynucleotide is delivered to the targeted site is not limiting. It can involve the use of a wide variety of mechanisms, including, for example, the use of a polynucleotide delivery matrix.

Small Interfering RNAs

The active agents used herein are polynucleotides that will cause RNA interference to suppress the expression of a PLOD2 gene, for example, by destruction of the mRNA. RNA interference involves the use of small double stranded RNA molecules termed siRNA, which complex with endonucleases to cleave a specific mRNA target.

Small interfering RNA (or siRNA) described herein, is a segment of double stranded RNA that is from 15 to 30 nucleotides in length. It is used to trigger a cellular reaction known as RNA interference. In RNA interference, double-stranded RNA is digested by an intracellular enzyme known as Dicer, producing siRNA duplexes. The siRNA duplexes bind to another intracellular enzyme complex, which is thereby activated to target whatever mRNA molecules are homologous (or complementary) to the siRNA sequence. The activated enzyme complex cleaves the targeted mRNA, destroying it and preventing it from being used to direct the synthesis of its corresponding protein product. Recent evidence suggests that RNA interference is an ancient, innate mechanism for not only defense against viral infection (many viruses introduce foreign RNA into cells) but also gene regulation at very fundamental levels. RNA interference has been found to occur in plants, insects, lower animals, and mammals, and has been found to be dramatically more effective than other gene silencing technologies, such as antisense or ribozyme technologies.

Used as a biotechnology technique, siRNA methodology involves introducing into cells (or causing cells to produce) short, double-stranded molecules of RNA similar to those that would be produced by the Dicer enzyme from an invading double-stranded RNA virus. The artificially-triggered RNA interference process then continues from that point.

To deliver a small interfering RNA to a subject, a preferred method involves introducing DNA encoding for the siRNA, rather than the siRNA molecules themselves, into target cells. The DNA sequence encoding for the particular therapeutic siRNA can be specified upon knowing (a) the sequence for a small and accessible portion of the target mRNA (available in public human genome databases, particularly the NCBI database), and (b) well-known scientific rules for how to specify DNA that will result in production of a corresponding RNA sequence when the DNA is transcribed by cells. The DNA sequence, once specified, can be constructed in the laboratory from synthetic molecules ordered from a laboratory supplier, and inserted using standard molecular biology methods into one of several alternative vectors for delivery of DNA to cells. Once delivered into the target cells, those cells will themselves produce the RNA that becomes the therapeutic siRNA, by transcribing the inserted DNA into RNA. The result will be that the cells themselves produce the siRNA that will silence the targeted gene (e.g., PLOD2 gene or a gene that encodes a protein involved in the production or processing of a TLH enzyme). The result will be a reduction of the amount of the targeted protein (e.g., TLH enzyme or a protein involved in the production or processing of a TLH enzyme) produced by the cell.

In accordance with the present invention, small interfering RNA against specific mRNAs produced in the targeted cells prevents the production of a TLH enzyme. Thus, also within the scope of the present invention is the use of specifically tailored vectors designed to deliver small interfering RNA directly to targeted cells. The success of the designed small interfering RNA is predicated on their successful delivery to the targeted cells.

Small interfering RNA molecules have been shown to be capable of targeting specific mRNA molecules in human cells. Small interfering RNA vectors can be constructed to transfect human cells and produce small interfering RNA that cause the cleavage of the target RNA and thereby interrupt production of the encoded protein.

A small interfering RNA vector of the present invention will prevent production of the pathogenic protein by suppressing production of a TLH enzyme itself or by suppressing production of a protein involved in the production or processing of a TLH enzyme. Repeated administration of the therapeutic agent to the subject may be required to accomplish the desired goal.

Exemplary siRNA sequences include those disclosed in the Example Section.

In the present invention, the small interfering RNA are targeted to complementary sequences in the mRNA sequence coding for the production of the target protein (e.g., TLH enzyme or a protein involved in the production or processing of a TLH enzyme), either within the actual protein coding sequence, or in the 5′ untranslated region or the 3′ untranslated region. After hybridization, the host enzymes guided by the siRNA are capable of cleavage of the mRNA sequence. Perfect or a very high degree of complementarity is typically needed for the small interfering RNA to be effective. A percent complementarity indicates the percentage of contiguous residues in a polynucleotide that can form hydrogen bonds (e.g., Watson-Crick base pairing) with a second polynucleotide (e.g., 5, 6, 7, 8, 9, 10 out of 10 being 50%, 60%, 70%, 80%, 90%, and 100% complementary). “Perfectly complementary” means that all the contiguous residues of a polynucleotide will hydrogen bond with the same number of contiguous residues in a second polynucleotide. However, it should be noted that single mismatches, or base-substitutions, within the siRNA sequence can substantially reduce the gene silencing activity of a small interfering RNA.

In preferred embodiments of the present invention, a small interfering RNA is 15 to 30 nucleotides in length. In particular embodiments, the polynucleotide is 15, 16, 17, 18, 19, 20, 21, 22, 23, 24, 25, 26, 27, 28, 29, or 30 nucleotides in length. In preferred embodiments the length of the siRNA sequence can be between 19-30 base pairs, and more preferably between 21 and 25 base pairs, and more preferably between 21 and 23 base pairs.

In a preferred embodiment, the invention provides a method for producing a class of nucleic acid-based gene inhibiting agents that exhibit a high degree of specificity for the RNA of a desired target. SiRNAs can be constructed in vitro or in vivo using appropriate transcription enzymes or expression vectors.

Examples of vectors for delivery of foreign DNA to mammalian cells include those well known to one of skill in the art, such as plasmids, viral or lenti vectors, particularly adeno-associated viral sectors. Other well-known techniques could also be used including electroporation.

SiRNAs can be constructed in vitro using DNA oligonucleotides. These oligonucleotides can be constructed to include an 8 base sequence complementary to the 5′ end of the T7 promoter primer included in the Silencer siRNA (Ambion Construction Kit 1620). Each gene specific oligonucleotide is annealed to a supplied T7 promoter primer, and a fill-in reaction with Klenow fragment generates a full-length DNA template for transcription into RNA. Two in vitro transcribed RNAs (one the antisense to the other) are generated by in vitro transcription reactions and then hybridized to each other to make double-stranded RNA. The double-stranded RNA product is treated with DNase (to remove the DNA transcription templates) and RNase (to polish the ends of the double-stranded RNA), and column purified to provide the siRNA that can be delivered and tested in cells.

Construction of siRNA vectors that express siRNAs within mammalian cells typically use an RNA polymerase III promoter to drive expression of a short hairpin RNA that mimics the structure of an siRNA. The insert that encodes this hairpin is designed to have two inverted repeats separated by a short spacer sequence. One inverted repeat is complementary to the mRNA to which the siRNA is targeted. A string of six consecutive thymidines added to the 3′ end serves as a pol III transcription termination site. Once inside the cell, the vector constitutively expresses the hairpin RNA. The hairpin RNA is processed into an siRNA that induces silencing of the expression of the target gene, which is called RNA interference (RNAi).

In most siRNA expression vectors described to date, one of three different RNA polymerase III (pol III) promoters is used to drive the expression of a small hairpin siRNA. These promoters include the well-characterized human and mouse U6 promoters and the human H1 promoter.

Delivery Compositions

The polynucleotides (i.e., siRNA molecules or DNA encoding siRNA molecules) can be embedded within, or otherwise associated with, a delivery matrix. This delivery matrix can be any or a wide variety of matrix materials. Examples of suitable systems are described, for example, in U.S. Pat. No. 5,962,427 (Goldstein et al.), as well as in Kyriakides et al., Molecular Therapy, 2, 842 (2001), and Bonadio et al., Nature Medicine, 5, 753 (1999).

The polynucleotides may be siRNA molecules that interfere with a PLOD2 gene and inhibit the translation of a TLH protein, or a protein involved in the production or processing of a TLH protein. Alternatively, the polynucleotide may encode siRNA molecules. Then interference with a PLOD2 gene and inhibition of the translation of a TLH enzyme is obtained when the polynucleotide is released from the delivery matrix and said polynucleotide is expressed in and the encoded siRNA molecule is delivered by the targeted cells and tissues.

The transferred DNA may be integrated into the genome of the target cell or not, as is possible with the use of this type of system.

The material that forms a polynucleotide delivery matrix is typically biocompatible. A material is generally “biocompatible” if it does not produce an adverse, allergic, or other undesired reaction when administered to a mammalian host. Such materials may be formed from both natural or synthetic materials.

Such materials may include, but are not limited to, biodegradable or non-biodegradable materials formulated into scaffolds that support cell attachment and growth, for example. Such materials may include synthetic polymers or naturally occurring proteins such as collagen, other extracellular matrix proteins, or other structural macromolecules. The material may be non-biodegradable in instances where it is desirable to leave permanent structures in the body; or biodegradable where the active agent (polynucleotide) is required only for a short duration of time.

In certain embodiments, such as for the polynucleotide delivery matrices, these materials may take the form of coatings, sponges, films, sheets, cuffs, implants, tubes, rods, microbeads, lyophilized components, gels, patches, powders or nanoparticles. In addition, for certain embodiments, such as for the polynucleotide delivery matrices, the materials used in the delivery of polynucleotides described herein can be designed to allow for sustained release (e.g., of the polynucleotide) over prolonged periods of time.

Depending on the embodiment in which it is used, physical and chemical characteristics, such as, e.g., biocompatibility, biodegradability, strength, rigidity, interface properties and even cosmetic appearance may be considered in choosing such a material, as is well known to those of skill in the art. Numerous examples of useful polymers are well known to those of skill in the art. It is to be understood that virtually any polymer that is now known or that will be later developed suitable for the sustained or controlled release of active agents (polynucleotides) may be employed in the present invention.

For certain embodiments, examples of useful non-degradable polymers include silicones, polyurethanes, silicone-urethane copolymers, polyimides, polysulphones, polyaryls, polyetheretherketones, polyetherketoneketones, polyacrylates, polymethacrylates, polymethylmethacrylates, polybutylmethacrylates, polytetrafluoroethylene, polyesters, polyolefins, polyethylenes, polypropylenes, polyamides, polyvinylchlorides, and epoxides.

For certain embodiments in which a biodegradable material (e.g., one that is capable of being reabsorbed into the body) is desired, suitable materials include, for example, synthetic organic polymers such as polyesters, polyanhydrides, polyethers, poly(orthoesters), poly(ether-esters), polyphosphazenes, poly(amino acids), polypeptides, and polyesteramides. More specifically, suitable biodegradable polymers materials are polylactic acid, polyglycolide, polylactic polyglycolic acid copolymers (“PLGA”), polycaprolactone (“PCL”), poly(dioxanone), poly(trimethylene carbonate) copolymers, polyglyconate, poly(propylene fumarate), poly(ethylene terephthalate), poly(butylene terephthalate), polyethyleneglycol, polycaprolactone copolymers, polyhydroxybutyrate, polyhydroxyvalerate, tyrosine-derived polycarbonates and any random or (multi-)block copolymers, such as bipolymer, terpolymer, quaterpolymer, etc., that can be polymerized from the monomers related to afore-listed homo- and copolymers.

Another particular group of suitable materials encompass the natural polymers. This group includes for example, polysaccharides, proteins and polypeptides, glycosaminoglycans, proteoglycans, collagen, elastin, hyaluronic acid, dermatan sulfate, chitin, chitosan, pectin, (modified) dextran, (modified) starch and modifications, mixtures or composites thereof.

A particularly suitable material is fibrous collagen, which may be lyophilized following extraction and partial purification from tissue and then sterilized. In addition, lattices made of collagen and glycosaminoglycan (GAG) may be used in the practice of the invention. At least 20 different forms of collagen have been identified and each of these collagens may be used in the practice of the invention.

Recombinant collagen may also be employed, as may be obtained from a collagen-expressing recombinant host cell, including bacterial, yeast, mammalian, plant and insect cells. The collagen used in the invention may, if so desired and applicable, be supplemented with additional minerals, such as calcium, e.g., in the form of calcium phosphate. Both native and recombinant type collagen may be supplemented by admixing, absorbing, or otherwise associating with, additional minerals in this manner.

Delivery

The present invention may provide one or more polynucleotides to the desired localized tissue region with various polynucleotide residence half-life times, generally of at least 24 hours. For example, the residence half-life of an active agent (polynucleotide) may be at least 1 day, at least 3 days, at least 1 week, at least 3 weeks, at least 4 weeks, at least 5 weeks, at least 6 weeks, or even longer.

A polynucleotide-containing composition can be designed to achieve constant or pulsed delivery to the localized tissue region at the site of the medical device. Pulsed delivery may be desirable in order to provide intermittent dosing of a polynucleotide to the local tissue region over time. For example, a combination of biodegradable polymers can be used that have differing degradation rates, and thus polynucleotide release rates. The composition may contain a homogeneous mixture of various biodegradable polymers, or the polymers may be utilized in a segmented fashion to achieve complex degradation profiles. The composition may also include various polymers to achieve zero-order, first-order, or other release. Also, the polynucleotide release timing may either be regular, e.g., initially and once weekly for several weeks, or it may be irregular, e.g., initially and then 3 days, 2 weeks, and 2 months apart.

A composition that includes one or more polynucleotides may be delivered to the localized tissue region via any suitable route, e.g., including, but not limited to, a subcutaneous, intradermal, intramuscular, intrathecal, intra-organ, intratumoral, intralesional, intravesicle, and intraperitoneal route of delivery. A “localized tissue region” will generally be a relatively small portion of the body, e.g., less than 10% by volume, and often less than 1% by volume.

For example, the localized tissue region will typically be on the order of no more than 500 cubic centimeters (cm³), often less than 100 cm³, and in many instances 10 cm³ or less. For some applications the localized tissue region will be 1 cm³ or less. However, in certain instances the localized tissue region may be a particularly large region, up to several liters.

The compositions may be delivered using, e.g., an implantable pump, a delivery catheter, needle injection, microneedle array, high-velocity particle implantation, or any other known method for introducing a composition into a localized tissue region (e.g., surgically implanted). Delivery to the localized tissue region may be in conjunction with image guiding techniques using, for example, ultrasound, MRI, real-time X-ray (fluoroscopy), etc.

The present invention also provides kits. For example, the kits may contain the components necessary for formation of a polynucleotide delivery matrix. In such cases the physician may combine the components to form the polynucleotide delivery matrices, which may then be used therapeutically by placement within the body. In one embodiment of the invention, polynucleotide delivery matrices may be used to coat surgical devices such as suture materials or other medical devices such as implants. In another embodiment of the invention, a sponge may be provided in the kit, which may then be impregnated with the desired polynucleotide (i.e., the active agent) by medical personnel prior to placement in the body.

### EXAMPLES

Objects and advantages of this invention are further illustrated by the following examples, but the particular materials and amounts thereof recited in these examples, as well as other conditions and details, should not be construed to unduly limit this invention.

### Example 1

### Evaluation of Inhibition of PLOD2 Expression in Human HeLa Cells by Means of siRNA

HeLa cells were one of the first human cells that are continuously grown in culture and are characterized by their ease of culturing and expansion. After having developed siRNA transfection protocols with HeLa cells we also showed that HeLa cells had an appropriate level of PLOD2 expression. This made them an appropriate candidate for testing the newly designed siRNA's against PLOD2.

Materials and Methods:

| | | | |
|---|---|---|---|
| siRNA #1: | sense sequence | 5′GGUCCUUGGUCAAGGAGAAtt 3′ | (SEQ ID NO:1) |
| | anti-sense sequence | 5′UUCUCCUUGACCAAGGACCtt 3′ | (SEQ ID NO:2) |
| siRNA #2: | sense sequence | 5′GGAGAAGAAUGGAGAGGUGtt 3′ | (SEQ ID NO:3) |
| | anti-sense sequence | 5′CACCUCUCCAUUCUUCUCCtt 3′ | (SEQ ID NO:4) |
| siRNA #3: | sense sequence | 5′GGUACAAUUGCUCUAUUGAtt 3′ | (SEQ ID NO:5) |
| | anti-sense sequence | 5′UCAAUAGAGCAAUUGUACCtt 3′ | (SEQ ID NO:6) |

Cell Culture: HeLa cells (ATCC) were cultured in Minimum Essential Medium with Earle's salts, L-glutamine, 0.1 millimolar (mM) non-essential amino acids, 1 mM sodium pyruvate, 10% fetal bovine serum (Gibco) at 37° C. and 5% CO₂.

Transfection Method: HeLa cells (5×10⁵ HeLa cells) were plated in a 25 square centimeters (cm²) culture flask containing 3 milliliter (mL) of normal growth medium one day before transfection so that they will be 50% confluent at the time of transfection. Before the transfection this normal growth medium was replaced with 7 mL growth medium without antibiotics.

The cells were transfected by using Oligofectamine transfection reagent (Invitrogen). For this transfection 84 microliters (μL) siRNA or water (control sample) was added to 1400 μL medium in 4 separate tubes, in 4 other tubes 84 μL Oligofectamine was added to 336 μL medium. All tubes were incubated at room temperature for 5 minutes. To the tubes containing the siRNA or water the contents of the Oligofectamine tubes was added and incubated for 20 minutes. From these mixtures 950 μL was added to the cell culture plates. Cells were cultured at 37° C. and 5% CO₂. RNA was isolated after 48 hours on incubation with 840 picomoles (pmol) siRNA (final concentration: 100 nanomolar (nM)).

Semi-quantification Method: The cells were harvested from the culture flasks with 1 mL of Trypsin-EDTA (0.25% Trypsin, 1 mM EDTA-4Na, Gibco) and the cell numbers were estimated by a cell count using a viability counter (Beckman coulter). Total RNA was isolated from the cells using the RNeasy mini kit (Qiagen) and gDNA was removed using an on column DNase treatment (30 minutes (min)). After isolation, absence of gDNA was confirmed by a PCR using the MCP-1 primer set. The A260 was measured to correct for the amount of RNA. Reverse transcription took place using the iScript cDNA synthesis kit (Bio-Rad). Subsequently, two Real Time PCR's were carried out using iQ SYBR Green supermix (Bio-Rad), one with the PLOD2 primer set and one with the GAPDH primer set. The PLOD2 Standard curve was generated using the control samples undiluted, 10× diluted, 100× diluted, and 1000× diluted. The undiluted sample was set at 100%, the others at respectively 10%, 1%, and 0.1%. The GAPDH standard curve was generated by a dilution series of a GAPDH control molecule, which has the same sequence as the PCR product.

Results:

Gel Electrophoresis: The quality of the RNA was checked by gel electrophoresis. High quality RNA has two bands, the 28S rRNA band and the 18S rRNA band. For high quality RNA the intensity of these bands should be 2:1; this was indeed confirmed.

Control PCR: To confirm absence of gDNA a real time PCR was carried out using SYBR Green. All RNA samples were confirmed to be free from gDNA.

Reverse Transcriptase Reaction: All RNA samples were diluted to the same concentration before continuing with the RT-reaction. RNA (3 micrograms (μg)) of each sample was reverse transcribed in a 60 μL reaction.

| | |
|---|---|
| RT-Reaction: | 12 μL Iscript supermix (Biorad, catalog number |
| | 170-8890) |
| | 3 μL Reverse transcriptase |
| | 33 μL H₂O |
| | 12 μL RNA in H₂O |
| | Incubation 5 minutes at 25° C., 30 minutes at 45° C. and |
| | 5 minutes at 85° C. |

PCR protocol PLOD2:

| | | |
|---|---|---|
| Cycle 1: (1X) | Step 1: 95.0° C. | for 10:00 |
| Cycle 2: (40X) | Step 1: 95.0° C. | for 00:30 |
| | Step 2: 63.3° C. | for 00:30 |
| | Step 3: 72.0° C. | for 00:30 |

Data collection and real-time analysis enabled.

| | | |
|---|---|---|
| Cycle 3: (1X) | Step 1: 72.0° C. | for 05:00 |
| Cycle 4: (1X) | Step 1: 50.0° C. | for 05:00 |
| Cycle 5: (225X) | Step 1: 50.0° C. | for 00:10 |

Increase set point temperature after cycle 2 by 0.2° C.
Melt curve data collection and analysis enabled.

| | | |
|---|---|---|
| Cycle 6: (1X) | Step 1: 4.0° C. | HOLD |

PCR protocol GAPDH:

| | | |
|---|---|---|
| Cycle 1: (1X) | Step 1: 95.0° C. | for 10:00 |
| Cycle 2: (40X) | Step 1: 95.0° C. | for 00:30 |
| | Step 2: 56.7° C. | for 00:30 |
| | Step 3: 72.0° C. | for 00:30 |

Data collection and real-time analysis enabled.

| | | |
|---|---|---|
| Cycle 3: (1X) | Step 1: 72.0° C. | for 05:00 |
| Cycle 4: (1X) | Step 1: 50.0° C. | for 05:00 |
| Cycle 5: (225X) | Step 1: 50.0° C. | for 00:10 |

Increase set point temperature after cycle 2 by 0.2° C.
Melt curve data collection and analysis enabled.

| | | |
|---|---|---|
| Cycle 6: (1X) | Step 1: 4.0° C. | HOLD |

The average threshold cycles for GAPDH were determined to be the same under all conditions tested (Tc approximately 15.0).

The results of PLOD2 expression in HeLa cells (Experiment 1) are shown in Table 1. The results are expressed as number of RT-PCR threshold cycles. Compared to control, untransfected fibroblasts, the difference in threshold cycles (ΔCt) was approximately 3-4 cycles.

TABLE 1
PLOD2 expression in HeLa cells (Experiment 1).

| Sample | Replicates threshold cycles | | | Average | sd |
|---|---|---|---|---|---|
| 1: siRNA #1 | 20.8 | 20.9 | 20.9 | 20.9 | 0.1 |
| 2: siRNA #2 | 21.7 | 21.2 | 21.5 | 21.5 | 0.3 |
| 3: siRNA #3 | 21.1 | 20.9 | 20.7 | 20.9 | 0.2 |
| 4: control | 17.7 | 17.6 | 17.8 | 17.7 | 0.1 |
| 5: siRNA #1 | 21.5 | 20.8 | 21.1 | 21.1 | 0.4 |
| 6: siRNA #2 | 20.7 | 20.4 | 20.1 | 20.4 | 0.3 |
| 7: siRNA #3 | 21.7 | 21.7 | 21.9 | 21.8 | 0.2 |
| 8: control | 17.8 | 17.1 | 17.8 | 17.5 | 0.4 |

In Table 2, the results listed in Table 1 are transformed and expressed as % relative PLOD2 expression. Compared to control, untransfected controls reduction of PLOD2 expression was approximately 90%.

TABLE 2

| | Average expression | Standard deviation |
|---|---|---|
| Sample | (%) | (%) |
| 1: siRNA #1 | 9.4 | 0.7 |
| 2: siRNA #2 | 6.2 | 1.1 |
| 3: siRNA #3 | 9.3 | 1.2 |
| 4: control | 88.9 | 5.3 |
| 5: siRNA #1 | 8.1 | 2.1 |
| 6: siRNA #2 | 13.5 | 2.5 |
| 7: siRNA #3 | 5.1 | 0.6 |
| 8: control | 103.6 | 31.5 |

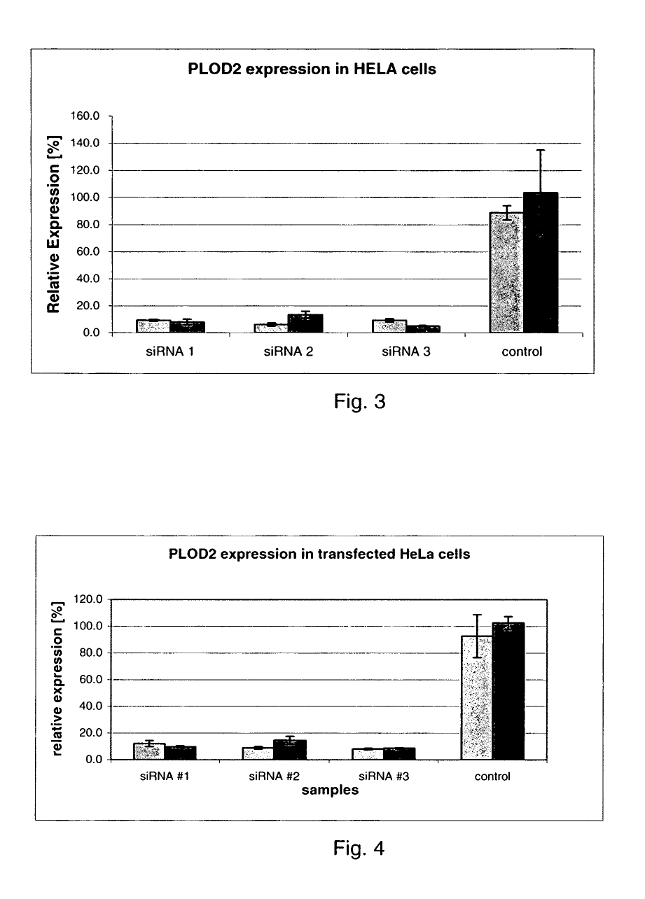

FIG. 3 gives a graphical representation of the results listed in Table 2.

In another independent experiment the above results were confirmed. Table 3 shows PLOD2 expression in HeLa cells Experiment 2). The results are expressed as number of RT-PCR threshold cycles. Compared to control, untransfected fibroblasts, the difference in threshold cycles (ΔCt) was approximately 3.5 cycles.

TABLE 3
PLOD2 expression in HeLa cells (Experiment 2).

| Sample | Replicates threshold cycles | | | Average | Std |
|---|---|---|---|---|---|
| 1: siRNA #1 | 20.8 | 20.3 | 20.6 | 20.5 | 0.3 |
| 2: siRNA #2 | 20.9 | 20.9 | 21.2 | 21.0 | 0.2 |
| 3: siRNA #3 | 21.1 | 21.3 | 21.1 | 21.1 | 0.1 |
| 4: control | 17.6 | 17.6 | 17.2 | 17.5 | 0.3 |
| 5: siRNA #1 | 20.8 | 20.7 | 21.0 | 20.8 | 0.1 |
| 6: siRNA #2 | 20.0 | 20.6 | 20.2 | 20.3 | 0.3 |
| 7: siRNA #3 | 21.1 | 21.0 | 20.9 | 21.0 | 0.1 |
| 8: control | 17.4 | 17.3 | 17.2 | 17.3 | 0.1 |

In Table 4, the results listed in Table 3 are transformed and expressed as % relative PLOD2 expression. Compared to control, untransfected controls reduction of PLOD2 expression was approximately 90%.

TABLE 4

| | Average expression | Standard deviation |
|---|---|---|
| Sample | (%) | (%) |
| 1: siRNA #1 | 12.2 | 2.2 |
| 2: siRNA #2 | 9.1 | 0.9 |
| 3: siRNA #3 | 8.1 | 0.6 |
| 4: control | 92.7 | 16.0 |
| 5: siRNA #1 | 9.8 | 0.8 |
| 6: siRNA #2 | 14.6 | 2.9 |
| 7: siRNA #3 | 8.7 | 0.5 |
| 8: control | 102.5 | 4.7 |

FIG. 4 gives a graphical representation of the results listed in Table 4.

Conclusion:

All siRNA's tested showed a significant reduction in PLOD2 gene expression. This reduction was approximately 90%.

### Example 2

### Quantification of PLOD2 Expression in Human Fibroblasts

Fibroblasts play a central role in the development of fibrous encapsulation of implants by way of the fact that fibroblasts are the main producers of collagen. In designing an anti-fibrous encapsulation therapy wherein the active agent suppresses the production and/or activity of a TLH enzyme it is thus deemed of significant importance to demonstrate the active agent's activity and efficacy using fibroblast cells. In this experiment three different siRNA designs, targeting PLOD2, were tested using human fibroblasts. RNA isolated from transfected human fibroblasts was quantified using Real Time RT-PCR and compared with RNA isolated from non-transfected cells.

Materials & Methods:

| | | | |
|---|---|---|---|
| siRNA #1: | sense sequence | 5′GGUCCUUGGUCAAGGAGAAtt 3′ | (SEQ ID NO:1) |
| | anti-sense sequence | 5′UUCUCCUUGACCAAGGACCtt 3′ | (SEQ ID NO:2) |
| siRNA #2: | sense sequence | 5′GGAGAAGAAUGGAGAGGUGtt 3′ | (SEQ ID NO:3) |
| | anti-sense sequence | 5′CACCUCUCCAUUCUUCUCCtt 3′ | (SEQ ID NO:4) |
| siRNA #3: | sense sequence | 5′GGUACAAUUGCUCUAUUGAtt 3′ | (SEQ ID NO:5) |
| | anti-sense sequence | 5′UCAAUAGAGCAAUUGUACCtt 3′ | (SEQ ID NO:6) |

Cell culture: Human fibroblasts (CCD-1077Sk cells, ATCC) were cultured in Iscoves modified Dulbecco's medium supplemented with 10% fetal bovine serum, 1% PSN antibiotics, 1% Fungizone antimycotics (Gibco) at 37° C. and 5% CO₂.

Transfection: 2×10⁵ fibroblasts were plated in a 25 cm² culture flask containing 7 mL of growth medium without antibiotics one day before transfection so that they will be 50% confluent at the time of transfection. The cells were transfected with 840 pmol siRNA (final concentration: 100 nM) using 14 μL Lipofectamine 2000 transfection reagent and 700 μL Opti-MEM I (Invitrogen). Growth medium was changed after 12 hours and RNA was isolated after 36 hours of incubation.

Semi-quantification: The cells were harvested from the culture flasks with 1 mL of Trypsin-EDTA (0.25% Trypsin, 1 mM EDTA-4Na, Gibco). Total RNA was isolated from the cells using the RNeasy mini kit (Qiagen) and gDNA was removed using an on column DNase treatment (30 minutes (min)). After isolation, absence of gDNA was confirmed by a PCR using the MCP-1 primerset. The A260 was measured to correct for the amount of RNA. Reverse transcription took place using the iScript cDNA synthesis kit (Bio-Rad). Subsequently, two Real Time PCR's were carried out using iQ SYBR Green supermix (Bio-Rad), one with PLOD2 primerset and one with the GAPDH primerset which is used as internal control. The GAPDH standard curve was generated by amplifying the following numbers of DNA control molecules (in triple) in a 25 μL reaction: 1×10¹⁰, 1×10⁹, 1×10⁸ . . . . to 1×10³. The DNA control molecule was chemically synthesized (Life Technologies) and has the same sequence as the PCR products. The PLOD2 standard curve was made by amplifying the cDNA of an untransfected control sample undiluted, 10× diluted, 100× diluted, and 1000× diluted. The undiluted sample was set at 100% expression the others at respectively 10%, 1%, and 0.1% expression.

The Real Time PCR's for LOX and COL1A2 were performed the same way as the Real Time PCR for PLOD2.

Results:

Gel electrophoresis: The quality of the RNA was checked by gel electrophoresis. High quality RNA has two bands, the 28S rRNA band and the 18S rRNA band. For high quality RNA the intensity of these bands should be 2:1; this was indeed confirmed.

Control PCR: To confirm absence of gDNA a real time PCR was carried out using SYBR Green. Besides the positive control, one sample crossed the threshold and gave a significant signal. Since this occurred very late in the reaction (cycle 38), it was neglected as non-significant.

Reverse Transcriptase Reaction: All RNA samples were diluted to the same concentration before continuing with the RT-reaction. The entire 40 μL of diluted sample (3 micrograms (μg) RNA) was reverse transcribed in a 60 μL reaction.

All siRNA's showed a significant reduction in the PLOD2 expression. All siRNA's reduced the PLOD2 expression in human fibroblasts with 90% (ΔCt approximately 4 cycles) as shown in Tables 5 and 6, and FIG. 3.

TABLE 5
Results of 2 evaluation studies (#A and #B) on
siRNA-based reduction of PLOD2 expression in fibroblasts.

| | Replicates RT-PCR | | | | | | Aver- | |
|---|---|---|---|---|---|---|---|---|
| Sample | threshold cycles (Ct) | | | | | | age | Sd |
| 1A: siRNA #1 | 24.6 | | 24.1 | | 23.9 | | 24.2 | 0.34 |
| 2A: siRNA #2 | 23.8 | | 23.8 | | 24.3 | | 23.9 | 0.27 |
| 3A: siRNA #3 | 24.7 | | 24.7 | | 24.6 | | 24.7 | 0.04 |
| 4A: control | 20.3 | | 20.6 | | 20.7 | | 20.5 | 0.21 |
| 1B: siRNA #1 | 24.1 | | 24.2 | | 24.3 | | 24.2 | 0.10 |
| 2B: siRNA #2 | 24.2 | | 24.1 | | 24.1 | | 24.1 | 0.07 |
| 3B: siRNA #3 | 24.7 | | 24.6 | | 24.4 | | 24.5 | 0.15 |
| 4B: control* | 20.7 | 20.2 | 20.8 | 20.4 | 0.37 | 19.8 | 20.4 | 0.37 |

*Control 4B was measured 6x; 3x as the undiluted standard and 3x as sample.

Results are expressed as number of RT-PCR threshold cycles. Compared to control, non-transfected fibroblasts the difference in threshold cycles (ΔCt) 4 cycles.

TABLE 6
Results of 2 evaluation studies (#A and #B) on siRNA-based
reduction of PLOD2 expression in fibroblasts.

| | Average | Standard deviation |
|---|---|---|
| Sample | (relative expression %) | (relative expression %) |
| 1A: siRNA #1 | 9 | 2.02 |
| 2A: siRNA #2 | 11 | 1.86 |
| 3A: siRNA #3 | 7 | 0.16 |
| 4A: control | 108 | 16.25 |
| 1B: siRNA #1 | 9 | 0.60 |
| 2B: siRNA #2 | 10 | 0.45 |
| 3B: siRNA #3 | 7 | 0.74 |
| 4B: control | 122 | 30.52 |

Results listed in Table 5 are transformed and expressed in Table 6 as a percentage (%) relative PLOD2 expression. Compared to control, non-transfected controls reduction of PLOD2 expression was 90%.

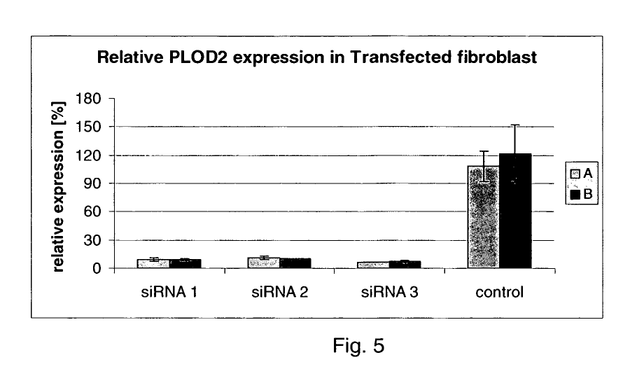

FIG. 5 gives a graphical representation of the results listed in Tables 5 and 6.

To demonstrate that the anti-PLOD2 activity of the used siRNAs was a specific effect and did not induce an inhibitory effect on other enzymes involved in collagen synthesis, the three siRNA's were also tested in the same fibroblast cells for their effect on the following genes: LOX (lysyl oxidase) and COL1A2 (collagen type 1, alpha 2). The results are shown in Tables 7 and 8.

TABLE 7
Effect of anti-PLOD2 siRNA's on Lox expression. Results
of 2 independent evaluation studies (#A and #B).

| | Replicates | | | | |
|---|---|---|---|---|---|
| Sample | threshold cycles | | | Average | sd |
| 1A: siRNA #1 | 18.8 | 18.7 | 18.1 | 18.8 | 0.12 |
| 2A: siRNA #2 | 19.4 | 19.2 | 19.6 | 19.4 | 0.22 |
| 3A: siRNA #3 | 19.2 | 19.2 | 19.2 | 19.2 | 0.02 |
| 4A: control | 19.2 | 18.6 | 18.9 | 18.9 | 0.27 |
| 1B: siRNA #1 | 18.8 | 19.2 | 17.9 | 19.0 | 0.33 |
| 2B: siRNA #2 | 19.3 | 19.2 | 19.2 | 19.2 | 0.06 |
| 3B: siRNA #3 | 19.3 | 18.8 | 18.8 | 19.0 | 0.27 |
| 4B: control | 19.1 | 18.9 | 18.9 | 19.0 | 0.10 |

The data presented in Table 7 shows that there was no significant difference in LOX expression due to siRNA treatment of the fibroblasts as can be derived from the fact that there is no difference observed in RT-PCR thresholds between the treatment and control groups.

TABLE 8
Effect of anti-PLOD2 siRNA's on COL1A2 expression. Results
of 2 independent evaluation studies (#A and #B).

| | Replicates | | | | |
|---|---|---|---|---|---|
| Sample | threshold cycles | | | Average | Sd |
| 1A: siRNA #1 | 16.5 | 15.5 | 16.6 | 16.6 | 0.07 |
| 2A: siRNA #2 | 16.2 | 16.2 | 16.5 | 16.3 | 0.21 |
| 3A: siRNA #3 | 16.1 | 16.1 | 16.1 | 16.1 | 0.01 |
| 4A: control | 15.3 | 15.7 | 15.8 | 15.6 | 0.23 |
| 1B: siRNA #1 | 16.0 | 15.9 | 15.8 | 15.9 | 0.11 |
| 2B: siRNA #2 | 15.8 | 15.9 | 15.7 | 15.8 | 0.10 |
| 3B: siRNA #3 | 15.8 | 15.9 | 15.9 | 15.9 | 0.03 |
| 4B: control | 15.7 | 15.6 | 15.7 | 15.7 | 0.06 |

The data presented in Table 8 shows that there was no significant difference in COL1A2 expression due to siRNA treatment of the fibroblasts as can be derived from the fact that there is no difference observed in RT-PCR thresholds between the treatment and control groups.

Conclusion:

The expression of lysyl oxidase (LOX) and collagen, type I, alpha 2 (COL1A2) was not affected by the siRNA's targeting PLOD2. This confirms the specificity of the 3 siRNA designs in hand for silencing of the PLOD2 gene.

### Example 3

### siRNA Specificity of PLOD2 Suppression in Human Fibroblasts

In this experiment three different siRNA designs were tested using human fibroblasts. Two siRNA designs, targeting PLOD2, (‘siRNA #1’ and ‘siRNA #3’) were previously demonstrated to strongly suppress PLOD2 expression. The third siRNA design (‘scrambled’) is a scrambled siRNA. The latter is included to verify specificity of the afore-determined siRNA-induced suppression of PLOD2. RNA isolated from transfected human fibroblasts was quantified using Real Time RT-PCR and compared with RNA isolated from non-transfected cells.

Materials & Methods:

| | | | |
|---|---|---|---|
| siRNA #1: | sense sequence | 5′GGUCCUUGGUCAAGGAGAAtt 3′ | (SEQ ID NO:1) |
| | anti-sense sequence | 5′UUCUCCUUGACCAAGGACCtt 3′ | (SEQ ID NO:2) |
| siRNA #3: | sense sequence | 5′GGUACAAUUGCUCUAUUGAtt 3′ | (SEQ ID NO:5) |
| | anti-sense sequence | 5′UCAAUAGAGCAAUUGUACCtt 3′ | (SEQ ID NO:6) |

scrambled: Scrambled siRNA was ordered at Ambion Inc. (catalog number 4611). Sequence is not known. The scrambled siRNA is claimed to have no homology to any known gene sequence from mouse, rat, or human.

Cell culture: Human fibroblasts (CCD-1077Sk cells, ATCC) were cultured in Iscoves modified Dulbecco's medium supplemented with 10% fetal bovine serum, 1% PSN antibiotics, 1% Fungizone antimycotics (Gibco) at 37° C. and 5% CO₂.

Transfection: 2×10⁵ fibroblasts were plated in a 25 cm² culture flask containing 7 mL of growth medium without antibiotics one day before transfection so that they will be 50% confluent at the time of transfection. The cells were transfected with 840 pmol siRNA (final concentration: 100 nM) diluted in 700 μL Opti-MEM I using 14 μL Lipofectamine 2000 transfection reagent diluted in another 700 μL Opti-MEM I (Invitrogen). Growth medium was changed after 12 hours and RNA was isolated after 36 hours of incubation.

Semi-quantification: The cells were harvested from the culture flasks with 1 mL of Trypsin-EDTA (0.25% Trypsin, 1 mM EDTA-4Na, Gibco). Total RNA was isolated from the cells using the RNeasy mini kit (Qiagen) and gDNA was removed using an on column DNase treatment (30 min). After isolation, absence of gDNA was confirmed by a PCR using the MCP-1 primerset. The A260 was measured to correct for the amount of RNA. Reverse transcription took place using the iScript cDNA synthesis kit (Bio-Rad). Subsequently, two Real Time PCR's were carried out using iQ SYBR Green supermix (Bio-Rad), one with PLOD2 primerset and one with the GAPDH primerset which is used as internal control. The GAPDH standard curve was generated by amplifying the following numbers of DNA control molecules (in triple) in a 25 μL reaction: 1×10¹⁰, 1×10⁹, 1×10⁸ . . . to 1×10³. The DNA control molecule was chemically synthesized (Life Technologies) and has the same sequence as the PCR product. The PLOD2 standard curve was made by amplifying the cDNA of an untransfected control sample undiluted, 10× diluted, 100× diluted, and 1000× diluted. The undiluted sample was set at 100% expression the others at respectively 10%, 1%, and 0.1% expression.

Results:

Gel electrophoresis: The quality of the RNA was checked by gel electrophoresis. High quality RNA has two bands, the 28S rRNA band and the 18S rRNA band. For high quality RNA the intensity of these bands should be 2:1; this was indeed confirmed.

Control PCR: To confirm absence of gDNA a real time PCR was carried out using SYBR Green. Besides the positive control, one sample crossed the threshold and gave a significant signal. Since this occurred very late in the reaction (cycle 38), it was neglected as non-significant.

Reverse Transcriptase Reaction: All RNA samples were diluted to the same concentration before continuing with the RT-reaction. The entire 40 μL of diluted sample (3 μg RNA) was reverse transcribed in a 60 μL reaction.

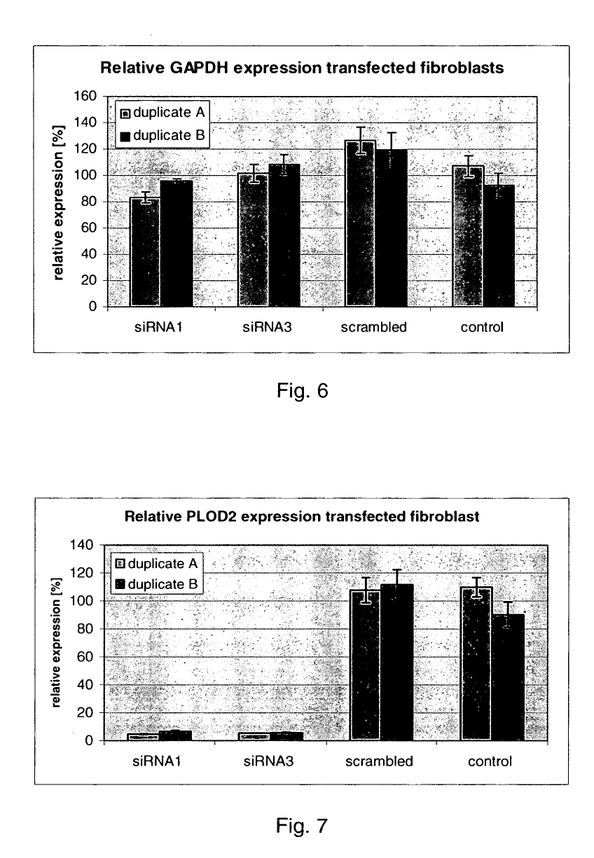

The designed siRNA's showed a significant reduction in the PLOD2 expression. siRNA #1 and siRNA #3 reduced the PLOD2 expression in human fibroblasts with 90% (ΔCt approximately 4 cycles). In contrast, the scrambled siRNA design did not show any effect on PLOD2 expression, as shown in FIGS. 6 and 7.

### Example 4

### Quantification of PLOD 2 in Transfected Human Skin Fibroblasts

Materials & Methods:

| | | | |
|---|---|---|---|
| siRNA #1: | sense sequence | 5′GGUCCUUGGUCAAGGAGAAtt 3′ | (SEQ ID NO:1) |
| | anti-sense sequence | 5′UUCUCCUUGACCAAGGACCtt 3′ | (SEQ ID NO:2) |
| siRNA #2: | sense sequence | 5′GGAGAAGAAUGGAGAGGUGtt 3′ | (SEQ ID NO:3) |
| | anti-sense sequence | 5′CACCUCUCCAUUCUUCUCCtt 3′ | (SEQ ID NO:4) |
| siRNA #3: | sense sequence | 5′GGUACAAUUGCUCUAUUGAtt 3′ | (SEQ ID NO:5) |
| | anti-sense sequence | 5′UCAAUAGAGCAAUUGUACCtt 3′ | (SEQ ID NO:6) |

Cell culture: Human fibroblasts (passage 10; donor 30/9) were isolated from skin tissue (Slot et al., J. Biol. Chem., 278(42):40967-72 (2003)). Medium with 4500 milligrams per liter (mg/L) Glucose, pyruvate and glutamax (GIBCO, ref 31966-021) supplemented with 10% heat-inactivated fetal bovine serum, and 1% Penicilin/streptomycin, antibiotics at 37° C. and 5% CO₂.

Transfection: 2×10⁵ cells were plated in a 6-wells plate containing 3 mL of medium without antibiotics one day before transfection. At the time of transfection the cells were 80-90% confluent. The cells were transfected with 840 pmol siRNA or block-it fluorescent oligo (Invitrogen) in 3.4 mL medium (final concentration: 250 nM) using 14 μL Lipofectamine 2000 and 700 μL Opti-MEM I (Invitrogen). Medium was changed after 14 hours and RNA was isolated after 40 hours of incubation.

Quantification: Cells were washed with PBS and lysated with 350 μL RLT buffer. Total RNA (30 μl) was isolated using the RNeasy mini kit (catalog number 74106;Qiagen). RNA (8.2 μL) was reverse transcribed into 20 μL cDNA (First Strand cDNA Synthesis kit; Roche ref. 1483188), diluted 10 times with milli Q water and subjected to real-time PCR amplification. Real-time PCR amplification of cDNA sequences was performed on 10 μL diluted cDNA for Plod 1, 2b, 3, a2 chain of collagen type 1 (Col1A2), α1 chain of collagen type 3 (Col3A1), Lysyl Oxidase (Lox), Proline 4-hydroxylase I (P4HA-1) and β-2-microglobulin (B2M) to standardize for differences in the total amount of cDNA. Each cDNA was amplified using specific primers and specific molecular beacons (Slot et al., J. Biol. Chem., 278(42):40967-72 (2003)) in a total reaction volume of 25 μL. PCR's were performed in an ABI PRISM 7700 sequence detection system and data were analyzed using sequence detector version 1.7 software.

Results:

TABLE 9
Real-time PCR data: Transfected human skin fibroblasts.

| | |
|---|---|
| cycli | fmol |

B2M expression

| | | |
|---|---|---|
| siRNA #1 a | 19.95 | 865.5 |
| siRNA #1 b | 20.19 | 755.9 |
| siRNA #2 a | 19.77 | 958.0 |
| siRNA #2 b | 20.98 | 484.1 |
| siRNA #3 a | 20.33 | 698.5 |
| siRNA #3 b | 18.39 | 2086.9 |
| control a | 19.07 | 1422.0 |
| control b | 18.93 | 1538.8 |
| GFP a | 19.15 | 1359.2 |
| GFP b | 19.87 | 905.5 |

Col1A2 expression

| | | |
|---|---|---|
| siRNA #1 a | 17.23 | 4609.0 |
| siRNA #1 b | 17.86 | 3110.9 |
| siRNA #2 a | 17.01 | 5287.1 |
| siRNA #2 b | 17.63 | 3591.0 |
| siRNA #3 a | 17.84 | 3150.0 |
| siRNA #3 b | 17.01 | 5287.1 |
| control a | 16.47 | 7405.4 |
| control b | 16.44 | 7545.3 |
| GFP a | 16.69 | 6455.6 |
| GFP b | 17.47 | 3968.0 |

Proline 4-Hydroxylase I (P4HA-1) expression

| | | |
|---|---|---|
| siRNA #1 a | 21.33 | 31.9 |
| siRNA #1 b | 21.27 | 33.0 |
| siRNA #2 a | 20.43 | 55.1 |
| siRNA #2 b | 21.34 | 31.7 |
| siRNA #3 a | 21.8 | 23.9 |
| siRNA #3 b | 21.98 | 21.5 |
| control a | 20.39 | 56.4 |
| control b | 20.2 | 63.3 |
| GFP a | 20.77 | 44.8 |
| GFP b | 20.48 | 53.4 |

PLOD1 expression

| | | |
|---|---|---|
| siRNA #1 a | 23.26 | 22.1 |
| siRNA #1 b | 23.6 | 16.9 |
| siRNA #2 a | 22.78 | 32.4 |
| siRNA #2 b | 23.62 | 16.6 |
| siRNA #3 a | 24.15 | 10.9 |
| siRNA #3 b | 23.2 | 23.2 |
| control a | 23.25 | 22.3 |
| control b | 23 | 27.2 |
| GFP a | 22.45 | 42.1 |
| GFP b | 23.04 | 26.3 |

PLOD2 expression

| | | |
|---|---|---|
| siRNA #1 a | 27.18 | 26.8 |
| siRNA #1 b | 28.01 | 17.3 |
| siRNA #2 a | 26.3 | 42.7 |
| siRNA #2 b | 28.25 | 15.3 |
| siRNA #3 a | 28.54 | 13.1 |
| siRNA #3 b | 27.06 | 28.6 |
| control a | 22.11 | 388.6 |
| control b | 22.5 | 316.4 |
| GFP a | 22.47 | 321.5 |
| GFP b | 23.04 | 238.0 |

Lysyl Oxidase (LOX) expression

| | | |
|---|---|---|
| siRNA #1 a | 18.82 | 3107.2 |
| siRNA #1 b | 19.46 | 2070.5 |
| siRNA #2 a | 18.25 | 4460.4 |
| siRNA #2 b | 19.31 | 2277.2 |
| siRNA #3 a | 19.24 | 2380.6 |
| siRNA #3 b | 18.15 | 4752.5 |
| control a | 17.41 | 7598.8 |
| control b | 17.21 | 8626.5 |
| GFP a | 17.95 | 5395.2 |
| GFP b | 18.61 | 3549.9 |

Col3A1 expression

| | | |
|---|---|---|
| siRNA #1 a | 18.07 | 172.3 |
| siRNA #1 b | 18.56 | 129.3 |
| siRNA #2 a | 17.18 | 290.4 |
| siRNA #2 b | 18.13 | 166.3 |
| siRNA #3 a | 18.35 | 146.2 |
| siRNA #3 b | 17.77 | 205.4 |
| control a | 17.43 | 250.8 |
| control b | 17.37 | 259.8 |
| GFP a | 17.24 | 280.3 |
| GFP b | 17.42 | 252.2 |

PLOD3 expression

| | | |
|---|---|---|
| siRNA #1 a | 24.19 | 149.5 |
| siRNA #1 b | 24.61 | 115.3 |
| siRNA #2 a | 23.9 | 179.0 |
| siRNA #2 b | 24.2 | 148.6 |
| siRNA #3 a | 24.27 | 142.3 |
| siRNA #3 b | 23.53 | 225.1 |
| control a | 22.06 | 559.6 |
| control b | 23.5 | 229.3 |
| GFP a | 23.81 | 189.2 |
| GFP b | 24.41 | 130.5 |

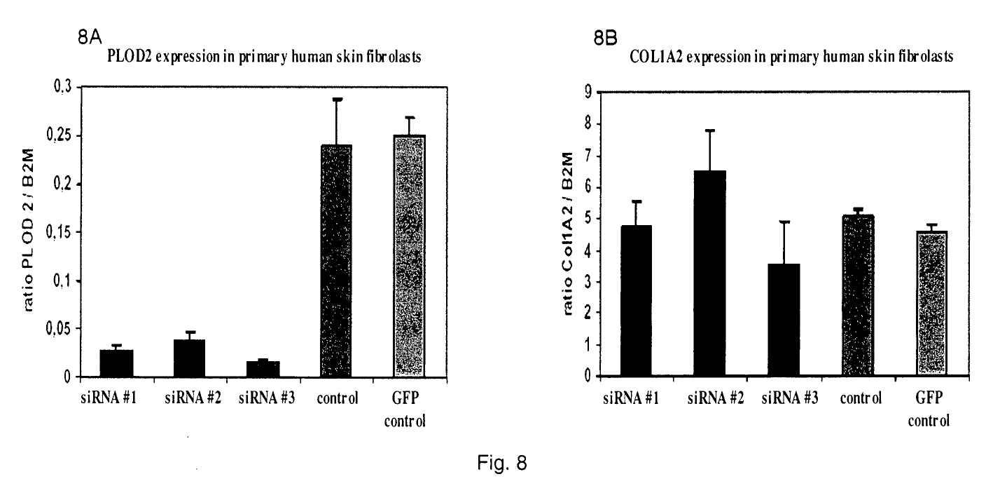

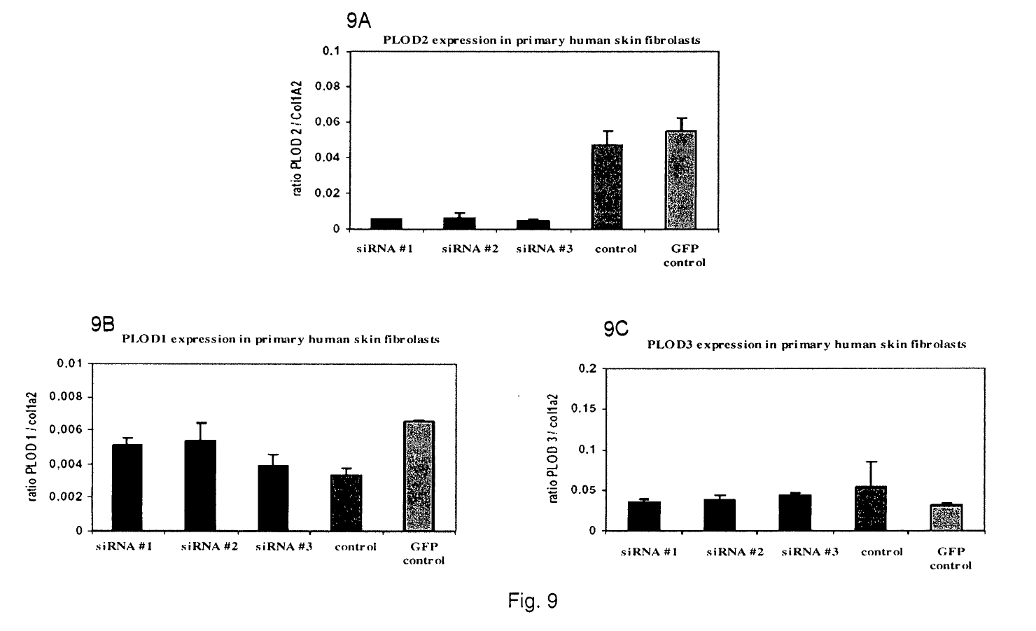

Also in the primary human skin fibroblast cells all siRNA's showed a significant reduction in the PLOD2 expression as can be seen in Table 9 and FIGS. 8 and 9. All siRNA's reduced the PLOD2 expression in human fibroblasts with 90% or more.

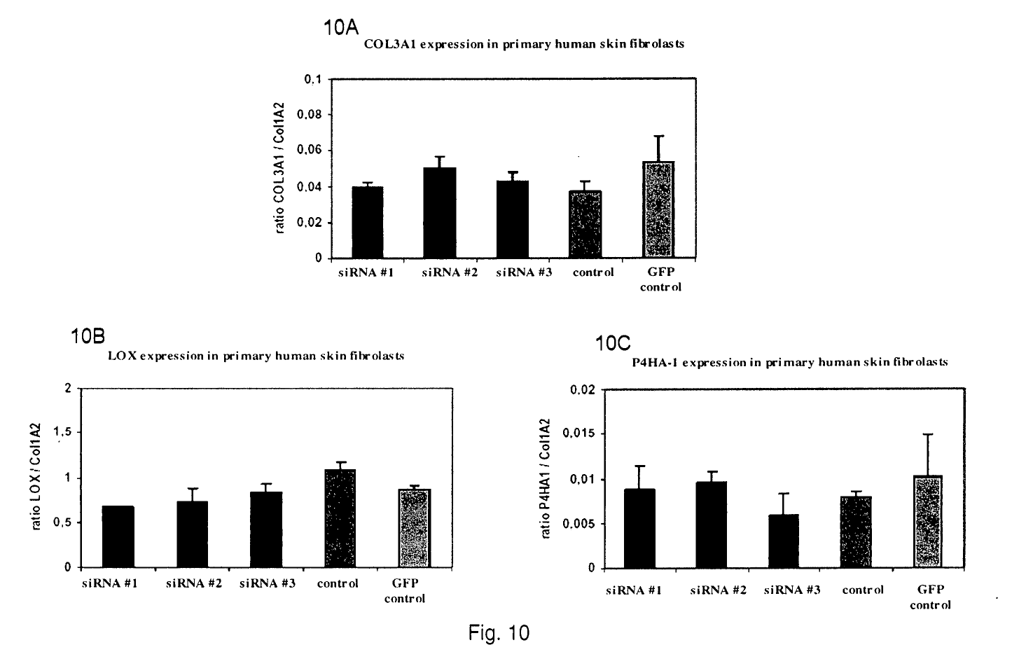

There was no significant difference in expression in any of the other genes due to siRNA treatment of the primary human skin fibroblasts as can be derived from the fact that there is no difference observed in RT-PCR threshold cycli between the treatment and control groups (Table 9 and FIGS. 8, 9, 10).

The results in FIGS. 8 and 9 demonstrate that siRNA induced suppression of PLOD2 does not interfere with the expression of COL1A2 nor any of the other collagen modifying enzymes.

Conclusion:

The effect of siRNA molecules on PLOD2 is very specific and is a promising new approach to prevent PLOD2-induced hydroxyallysine crosslinking of collagen.

### Example 5

### Quantification of PLOD 2 in Transfected Rat Skin Fibroblasts

The aim of this experiment is to test the performance of rat PLOD2 siRNA in rat fibroblasts.

Materials & Methods: siRNA were Synthesized by Ambion Inc. siRNA Sequences Toward Rat PLOD2:

| | | |
|---|---|---|
| Nr 1: siRNA ID:47463 | Sense seq: 5′ GCAGAUAAGUUAUUAGUCAtt 3′ | (SEQ ID NO:7) |
| | Anti-sense 5′ UGACUAAUAACUUAUCUGCtg 3′ | (SEQ ID NO:8) |
| Nr 2: siRNA ID:47549 | Sense seq: 5′ GAAAACGAUGGAUUCCACAtt 3′ | (SEQ ID NO:9) |
| | Anti-sense 5′ UGUGGAAUCCAUCGUUUUCtt 3′ | (SEQ ID NO:10) |
| Nr 3: siRNA ID:47630 | Sense seq: 5′ GAUUUAUGAAUUCAGCCAAtt 3′ | (SEQ ID NO:11) |
| | Anti-sense 5′ UUGGCUGAAUUCAUAAAUCtg 3′ | (SEQ ID NO:12) |

Cell culture: Rat fibroblasts (passage +6; ATCC 1213-CRL) were cultured in DMEM medium with 4500 milligrams per liter (mg/L) glucose, pyruvate and glutamax (GIBCO No. 31966-021) supplemented with 10% heat-inactivated fetal bovine serum, and 1% penicillin/streptomycin antibiotics at standard culture conditions (polystyrene culture wells; 37° C.; 5% CO₂).

Transfection: 5×10⁵ cells were plated in a 6-wells plate containing 3 mL of medium without antibiotics one day before transfection. At the time of transfection the cells were 80-90% confluent. The cells were transfected with 840 pmol siRNA or block-it fluorescent oligo (Invitrogen) in 3.4 mL medium (final concentration: 250 nanomolar (nM)) using 14 μL Lipofectamine 2000 and 700 μL Opti-MEM I (Invitrogen). Medium was changed after 8 hours and RNA was isolated after 40 hours of incubation.

Quantification: Cells were washed with PBS and lysated with 350 μL RLT buffer. Total RNA (30 μL) was isolated using the RNeasy mini kit (Qiagen No. 74106). RNA (8.2 μL) was reverse transcribed into 20 μL cDNA (First Strand cDNA Synthesis kit; Roche No. 1483188), diluted 10 times with Milli-Q water and subjected to real-time PCR amplification. Real-time PCR amplification of cDNA sequences was performed on 10 μL diluted cDNA for PLOD 1, 2, 3, the α2-chain of collagen type I (Col1A2), the α1-chain of collagen type III (Col3A1), lysyl oxidase (LOX), prolyl-4-hydroxylase 1 (P4HA1) and β2-microglobulin (B2M). The latter gene was used to standardize for differences in the total amount of cDNA. Each cDNA was amplified using specific primers and specific molecular beacons (designed for the rat genes) in a total reaction volume of 25 μL. Real-time PCR reactions were performed in an ABI PRISM 7700 sequence detection system and data were analyzed using Sequence detector version 1.7 software.

TABLE 10
Real-time PCR data: Transfected rat skin fibroblasts.

| | | | | |
|---|---|---|---|---|
| cycli | cycli | fmol | fmol | mean |

Rat B2M

| | | | | | |
|---|---|---|---|---|---|
| control a | 19.36 | 18.90 | 1091.98 | 1462.34 | 1277.16 |
| control b | 19.59 | 19.32 | 943.62 | 1120.06 | 1031.84 |
| siRNA nr 1a | 19.93 | 19.65 | 760.42 | 908.35 | 834.38 |
| siRNA nr 1b | 19.75 | 19.30 | 852.47 | 1134.38 | 993.43 |
| siRNA nr 2a | 19.56 | 19.29 | 961.77 | 1141.60 | 1051.68 |
| siRNA nr 2b | 19.42 | 18.92 | 1051.16 | 1443.89 | 1247.53 |
| siRNA nr 3a | 18.80 | 18.96 | 1558.19 | 1407.68 | 1482.93 |
| siRNA nr 3b | 19.49 | 19.26 | 1005.47 | 1163.55 | 1084.51 |
| block-it GFP | 18.62 | 18.45 | 1746.83 | 1945.91 | 1846.37 |
| Rat PLOD 2 | | | | | |
| control a | 20.54 | 20.86 | 651.48 | 521.14 | 586.31 |
| control b | 20.69 | 20.63 | 586.76 | 611.84 | 599.30 |
| siRNA nr 1a | 24.03 | 24.12 | 57.09 | 53.62 | 55.35 |
| siRNA nr 1b | 24.10 | 23.62 | 54.37 | 75.99 | 65.18 |
| siRNA nr 2a | 23.09 | 23.18 | 109.99 | 103.30 | 106.64 |
| siRNA nr 2b | 23.35 | 23.29 | 91.74 | 95.67 | 93.71 |
| siRNA nr 3a | 23.06 | 22.49 | 112.32 | 167.16 | 139.74 |
| siRNA nr 3b | 23.99 | 24.09 | 58.71 | 54.75 | 56.73 |
| block-it GFP | 20.62 | 20.52 | 616.12 | 660.63 | 638.38 |
| Rat PLOD 3 | | | | | |
| control a | 18.34 | 18.72 | 253.94 | 198.61 | 226.27 |
| control b | 18.55 | 18.75 | 221.69 | 194.79 | 208.24 |
| siRNA nr 1a | 19.78 | 19.59 | 100.07 | 113.15 | 106.61 |
| siRNA nr 1b | 19.12 | 19.20 | 153.34 | 145.61 | 149.47 |
| siRNA nr 2a | 19.12 | 18.91 | 153.34 | 175.65 | 164.49 |
| siRNA nr 2b | 19.53 | 19.24 | 117.63 | 141.89 | 129.76 |
| siRNA nr 3a | 19.66 | 19.20 | 108.14 | 145.61 | 126.87 |
| siRNA nr 3b | 20.10 | 19.49 | 81.36 | 120.71 | 101.03 |
| block-it GFP | 18.58 | 18.46 | 217.43 | 234.98 | 226.20 |
| Rat Col 3A1 | | | | | |
| control a | 15.31 | 15.49 | 62295.91 | 54009.07 | 58152.49 |
| control b | 15.93 | 15.74 | 38100.31 | 44296.10 | 41198.21 |
| siRNA nr 1a | 16.29 | 16.66 | 28638.01 | 21355.66 | 24996.84 |
| siRNA nr 1b | 16.06 | 16.07 | 34368.13 | 34096.66 | 34232.40 |
| siRNA nr 2a | 16.09 | 16.16 | 33560.14 | 31747.93 | 32654.04 |
| siRNA nr 2b | 16.20 | 16.43 | 30756.67 | 25628.67 | 28192.67 |
| siRNA nr 3a | 17.51 | 17.44 | 10883.51 | 11504.75 | 11194.13 |
| siRNA nr 3b | 16.98 | 17.18 | 16569.27 | 14139.12 | 15354.20 |
| block-it GFP | 15.92 | 15.96 | 38403.66 | 37204.58 | 37804.12 |
| Rat Col 1A2 | | | | | |
| control a | 14.71 | 14.86 | 34775.71 | 31160.20 | 32967.96 |
| control b | 15.21 | 15.07 | 24118.86 | 26721.09 | 25419.98 |
| siRNA nr 1a | 15.93 | 16.29 | 14240.10 | 10941.86 | 12590.98 |
| siRNA nr 1b | 15.42 | 15.87 | 20682.87 | 14879.33 | 17781.10 |
| siRNA nr 2a | 15.55 | 15.59 | 18805.80 | 18263.25 | 18534.53 |
| siRNA nr 2b | 15.89 | 15.75 | 14663.13 | 16245.16 | 15454.14 |
| siRNA nr 3a | 16.40 | 16.38 | 10095.53 | 10244.38 | 10169.95 |
| siRNA nr 3b | 16.31 | 16.34 | 10782.87 | 10548.71 | 10665.79 |
| block-it GFP | 15.42 | 15.43 | 20682.87 | 20532.05 | 20607.46 |
| Rat PLOD 1 | | | | | |
| control a | 20.60 | 20.02 | 108.25 | 168.98 | 138.61 |
| control b | 20.58 | 21.00 | 109.92 | 79.62 | 94.77 |
| siRNA nr 1a | 20.90 | 21.30 | 85.98 | 63.24 | 74.61 |
| siRNA nr 1b | 20.94 | 20.73 | 83.38 | 97.97 | 90.67 |
| siRNA nr 2a | 20.81 | 20.85 | 92.13 | 89.34 | 90.74 |
| siRNA nr 2b | 20.64 | 20.68 | 104.97 | 101.80 | 103.39 |
| siRNA nr 3a | 20.31 | 20.63 | 135.25 | 105.78 | 120.51 |
| siRNA nr 3b | 20.92 | 20.95 | 84.67 | 82.74 | 83.70 |
| block-it GFP | 20.40 | 20.11 | 126.22 | 157.69 | 141.95 |
| Rat LOX | | | | | |
| control a | 18.84 | 18.67 | 9091.09 | 10082.82 | 9586.95 |
| control b | 18.59 | 18.85 | 10586.26 | 9035.88 | 9811.07 |
| siRNA nr 1a | 19.58 | 19.55 | 5792.69 | 5899.51 | 5846.10 |
| siRNA nr 1b | 19.40 | 19.81 | 6463.86 | 5035.51 | 5749.68 |
| siRNA nr 2a | 20.46 | 20.02 | 3389.33 | 4430.95 | 3910.14 |
| siRNA nr 2b | 19.60 | 19.62 | 5722.56 | 5653.27 | 5687.92 |
| siRNA nr 3a | 18.68 | 18.97 | 10021.60 | 8399.04 | 9210.32 |
| siRNA nr 3b | 19.60 | 19.81 | 5722.56 | 5035.51 | 5379.04 |
| block-it GFP | 18.91 | 18.59 | 8711.65 | 10586.26 | 9648.95 |
| Rat P4HA-1 | | | | | |
| control a | 20.10 | 20.30 | 6230.70 | 5569.71 | 5900.20 |
| control b | 20.06 | 20.15 | 6372.02 | 6058.44 | 6215.23 |
| siRNA nr 1a | 21.48 | 21.00 | 2873.94 | 3761.56 | 3317.75 |
| siRNA nr 1b | 21.13 | 21.42 | 3497.12 | 2972.28 | 3234.70 |
| siRNA nr 2a | 21.12 | 20.89 | 3516.78 | 4000.88 | 3758.83 |
| siRNA nr 2b | 21.30 | 20.81 | 3179.16 | 4184.44 | 3681.80 |
| siRNA nr 3a | 20.57 | 20.19 | 4787.20 | 5924.06 | 5355.63 |
| siRNA nr 3b | 21.28 | 21.03 | 3215.01 | 3698.81 | 3456.91 |
| block-it GFP | 20.04 | 19.80 | 6443.89 | 7372.13 | 6908.01 |

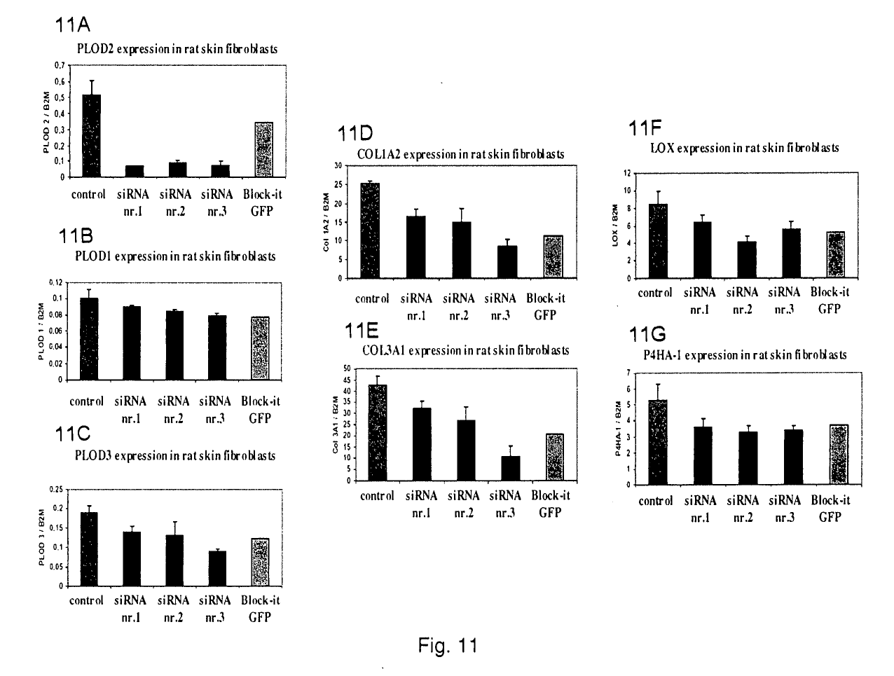

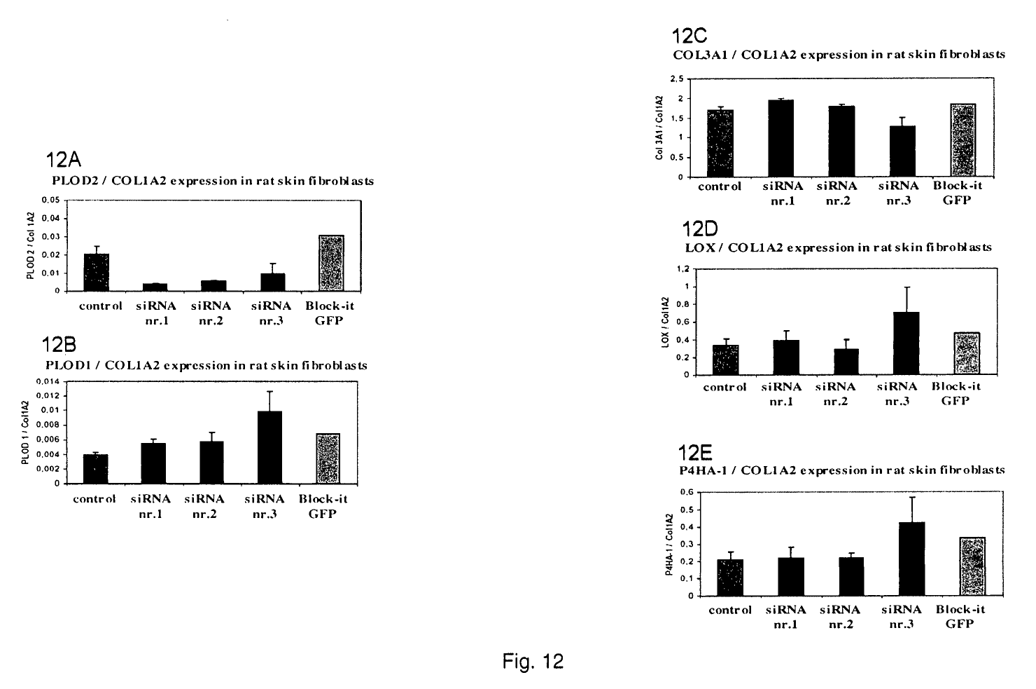

In rat skin fibroblasts PLOD2 expression is strongly suppressed with all three siRNA's oligonucleotides as can be seen in Table 10 and FIGS. 11 and 12.

Conclusion:

There is a good suppression of mRNA levels of PLOD2 with all three siRNA sequences. The siRNA has no effects on mRNA levels of PLOD1, PLOD3, lysyl oxidase (LOX), prolyl-4-hydroxylase-1 (P4HA-1), collagen type I (COL1A2) and collagen type III (COL3A1).

The complete disclosures of all patents, patent applications, publications, and nucleic acid and protein database entries, including for example GenBank accession numbers and EMBL accession numbers, that are cited herein are hereby incorporated by reference as if individually incorporated. Various modifications and alterations of this invention will become apparent to those skilled in the art without departing from the scope and spirit of this invention, and it should be understood that this invention is not to be unduly limited to the illustrative embodiments set forth herein.

What is claimed is:

1. A method of reducing or preventing fibrotic tissue formation in a subject, the method comprising delivering a polynucleotide that suppresses the production and/or activity of a TLH enzyme in collagen-producing cells, wherein the polynucleotide comprises an siRNA molecule or DNA encoding an siRNA molecule, wherein the siRNA molecule interferes with a PLOD2 gene and inhibits the translation of a TLH enzyme, or interferes with a gene that encodes a protein involved in the production or processing of a TLH enzyme.

2. The method of claim 1 wherein the polynucleotide is embedded in a polynucleotide delivery matrix.

3. The method of claim 2 wherein the polynucleotide delivery matrix comprises an siRNA molecule that interferes with a PLOD2 gene and inhibits the translation of a TLH enzyme or interferes with a gene encoding a protein involved in the production or processing of a TLH enzyme.

4. The method of claim 2 wherein the polynucleotide delivery matrix comprises DNA encoding an siRNA molecule that interferes with a PLOD2 gene and inhibits the translation of a TLH enzyme or interferes with a gene encoding a protein involved in the production or processing of a TLH enzyme.

5. The method of claim 1 wherein the polynucleotide is delivered to the desired site using a delivery method selected from the group consisting of a subcutaneous, intradermal, intramuscular, intrathecal, intra-organ, intratumoral, intralesional, intravesicle, and intraperitoneal method.

6. The method of claim 1 wherein the polynucleotide is delivered to a localized tissue region.

7. The method of claim 6 wherein the polynucleotide is delivered to a localized tissue region using a device selected from the group consisting of an implantable pumps, a delivery catheter, a needle, a microneedle array, and a device for high-velocity particle implantation.

8. The method of claim 1 wherein the fibrotic tissue formation is associated with heart rhythm disorder, heart failure, valve disease, vascular disease, diabetes, neurological diseases and disorders, or surgery.

9. The method of claim 1 wherein the fibrotic tissue formation occurs in myocardial infarct related fibrosis, cardiac fibrosis, valvular stenosis, intimal hyperplasia, diabetic ulcers, peridural fibrosis, perineural fibrosis, radiation induced fibrosis, macular degeneration, or rhino-sinusitis related fibrosis.


SEQUENCE LISTING (12 sequences)

SEQ ID NO: 1 - DNA, 21 bp, artificial
  Small interfering RNA in which bases 1-19 are ribonucleic acids, and bases 20 and 21 are deoxynucleic acids.
gguccuuggucaaggagaatt

SEQ ID NO: 2 - DNA, 21 bp, artificial
  Small interfering RNA in which bases 1-19 are ribonucleic acids, and bases 20 and 21 are deoxynucleic acids.
uucuccuugaccaaggacctt

SEQ ID NO: 3 - DNA, 21 bp, artificial
  Small interfering RNA in which bases 1-19 are ribonucleic acids, and bases 20 and 21 are deoxynucleic acids.
ggagaagaauggagaggugtt

SEQ ID NO: 4 - DNA, 21 bp, artificial
  Small interfering RNA in which bases 1-19 are ribonucleic acids, and bases 20 and 21 are deoxynucleic acids.
caccucuccauucuucucctt

SEQ ID NO: 5 - DNA, 21 bp, artificial
  Small interfering RNA in which bases 1-19 are ribonucleic acids, and bases 20 and 21 are deoxynucleic acids.
gguacaauugcucuauugatt

SEQ ID NO: 6 - DNA, 21 bp, artificial
  Small interfering RNA in which bases 1-19 are ribonucleic acids, and bases 20 and 21 are deoxynucleic acids.
ucaauagagcaauuguacctt

SEQ ID NO: 7 - DNA, 21 bp, artificial
  Small interfering RNA in which bases 1-19 are ribonucleic acids, and bases 20 and 21 are deoxynucleic acids.
gcagauaaguuauuagucatt

SEQ ID NO: 8 - DNA, 21 bp, artificial
  Small interfering RNA in which bases 1-19 are ribonucleic acids, and bases 20 and 21 are deoxynucleic acids.
ugacuaauaacuuaucugctg

SEQ ID NO: 9 - DNA, 21 bp, artificial
  Small interfering RNA in which bases 1-19 are ribonucleic acids, and bases 20 and 21 are deoxynucleic acids.
gaaaacgauggauuccacatt

SEQ ID NO: 10 - DNA, 21 bp, artificial
  Small interfering RNA in which bases 1-19 are ribonucleic acids, and bases 20 and 21 are deoxynucleic acids.
uguggaauccaucguuuuctt

SEQ ID NO: 11 - DNA, 21 bp, artificial
  Small interfering RNA in which bases 1-19 are ribonucleic acids, and bases 20 and 21 are deoxynucleic acids.
gauuuaugaauucagccaatt

SEQ ID NO: 12 - DNA, 21 bp, artificial
  Small interfering RNA in which bases 1-19 are ribonucleic acids, and bases 20 and 21 are deoxynucleic acids.
uuggcugaauucauaaauctg